(19) United States
(12) Patent Application Publication
(10) Pub. No.: US 2015/0299345 A1
(43) Pub. Date: Oct. 22, 2015

(54) ANTIBACTERIAL MONOMERS, ANTIBACTERIAL RESINS AND DENTAL COMPOSITES COMPRISING THE ANTIBACTERIAL RESINS

(71) Applicants: Huakun XU, Frederick, MD (US); Michael WEIR, Silver Spring, MD (US)

(72) Inventors: Huakun XU, Frederick, MD (US); Michael WEIR, Silver Spring, MD (US)

(73) Assignee: UNIVERSITY OF MARYLAND, BALTIMORE, Baltimore, MD (US)

(21) Appl. No.: 14/436,377

(22) PCT Filed: Sep. 19, 2013
(86) PCT No.: PCT/US2013/060679

§ 371 Date: Apr. 16, 2015

Related U.S. Application Data
(60) Provisional application No. 61/714,531, filed on Oct. 16, 2012.

(51) Int. Cl. C08F 20/34; A61Q 11/00; A61K 8/02; A61K 8/26; A61K 8/24; A61K 8/81; A61K 8/19

(52) U.S. Cl. CPC C08F 20/34; A61K 8/8152; A61Q 11/00; A61K 8/19; A61K 8/26; A61K 8/24; A61K 8/0241; A61K 2800/5426; A61K 2800/413

(57) ABSTRACT

Provided herein are antibacterial monomers, antibacterial resins comprising a resin and the monomers, and dental composites comprising the antibacterial resins and a filler, and methods of making the same. The dental composite can optionally contain amorphous calcium phosphate nanoparticles. The dental composites display strongly antibacterial properties, increased calcium and phosphate ion release, and improved mechanical properties.

### STATEMENT REGARDING FEDERALLY SPONSORED RESEARCH

This invention was made with government support under Grant Number DE017974 awarded by the National Institutes of Health. The government has certain rights in the invention.

### BACKGROUND

Tooth caries are the result of a dietary carbohydrate-modified bacterial infectious disease, one of the most common bacterial infections in humans (Loesche, 1986; van Houte, 1994; Featherstone, 2000). The basic mechanism of dental caries is demineralization, or mineral loss, through attack by acid generated by bacteria (Featherstone, 2004; Deng, 2004; Totiam et al., 2007). Therefore, acidogenic bacteria growth and biofilm formation are responsible for dental caries (Loesche, 1986; van Houte, 1994; Zero, 1995; Featherstone, 2000; Deng et al., 2005; Cenci et al., 2009). Plaque formation has been described to have three steps: pellicle formation, bacteria colonization, and biofilm maturation (Burne, 1998). In the initial stage, a proteinaceous film called pellicle forms on the tooth surface with adsorbed components from saliva, mucosa, and bacteria (Carlen et al., 2001). Bacteria then adhere and colonize on this surface to grow into a biofilm, which is a heterogeneous structure consisting of clusters of various types of bacteria embedded in an extracellular matrix (Stoodley et al., 2008). Cariogenic bacteria such as Streptococcus mutans (S. mutans) and lactobacilli in the plaque can take nutrients from carbohydrates and produce organic acids. Acid production causes demineralization to the tooth structure beneath the biofilm.

Resin composites have been increasingly used for tooth cavity restorations because of their aesthetics, direct-filling capability, and enhanced performance (Ferracane, 1995; Bayne et al., 1998; Lim et al., 2002; Ruddell et al., 2002; Watts et al., 2003; Drummond, 2008). While there has been significant improvement in resin compositions, filler types, and cure conditions since their introduction (Ruddell et al., 2002; Imazato, 2003; Drummond and Bapna, 2003; Watts et al., 2003; Lu et al., 2005; Xu X et al., 2006; Kramer et al., 2006), secondary caries formation and bulk fracture remain challenges to the use of resins (Sarrett, 2005; Sakaguchi, 2005).

Furthermore, resin composites in general do not prevent secondary caries because they do not hinder bacteria colonization and plaque formation. In fact, several studies have indicated that composites have a greater accumulation of bacteria and plaque than other restorative materials (Svanberg et al., 1990; Imazato et al., 1994; Takahashi et al., 2004). Indeed, caries at the restoration margins are a frequent reason for replacing existing restorations (Mjör et al., 2000), accounting for 50-70% of all restorations (Deligeorgi et al., 2001; Frost, 2002). In addition, frequent occurrence of gingivitis was reported when composites were placed at the subgingival area (van Dijken et al., 1991). Replacement dentistry costs $5 billion/year in the U.S. (Jokstad et al., 2001).

Therefore, there is a need for mechanically-strong composites that can inhibit the adherence and growth of bacteria, and thereby prevent secondary caries formation.

### SUMMARY

In a first embodiment, the present invention is directed to new antibacterial monomers that differ based on the alkyl chain length. The monomers include the following:

dimethylamino propyl methacrylate (DMAPM),

dimethylamino hexyl methacrylate (DMAHM),

dimethylamino heptyl methacrylate (DMAHPM),

dimethylamino octyl methacrylate (DMAOM),

dimethylamino nonyl methacrylate (DMANM),

dimethylamino decyl methacrylate (DMADM),

dimethylamino undecyl methacrylate (DMAUDM),

dimethylamino dodecyl methacrylate (DMADDM),

dimethylamino tridecyl methacrylate (DMATDM),

dimethylamino tetradecyl methacrylate (DMATTDM),

dimethylamino pentadecyl methacrylate (DMAPDM),

dimethylamino hexadecyl methacrylate (DMAHDM),

dimethylamino heptadecyl methacrylate (DMAHPDM),

dimethylamino octadecyl methacrylate (DMAODM),

dimethylamino nonadecyl methacrylate (DMANDM),

dimethylamino icosyl methacrylate (DMAIOM),

dimethylamino henicosyl methacrylate (DMAHOM), and

dimethylamino docosyl methacrylate (DMADOM).

In a second embodiment, the present invention is directed to antibacterial resins comprising a resin and an antibacterial monomer of the present invention. The resin is one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer. In certain aspects, the resin is a 1:1 mass ratio of bis-GMA and TEGDMA.

The antibacterial resins may comprise one or more than one of the antibacterial monomers of the invention. The combined amount of the one or more antibacterial monomers in the antibacterial resins of the present invention is a mass fraction of from about 0.5% to about 50% of the antibacterial resin. In certain aspects, the antibacterial resin comprises a combined amount of from about 1% to about 25%, about 2.5% to about 20%, about 5% to about 15%, or about 7.5% to about 12.5% by mass of one or more of the antibacterial monomers. In certain aspects, the antibacterial resin comprises a combined amount of about 2.5%, about 5%, about 7.5% or about 10% by mass of one or more of the antibacterial monomers.

The antibacterial resins may further comprise one or more additional antibacterial agents, including, but not limited to, quaternary ammonium salts (QAS), silver-containing nanoparticles (NAg), chlorhexidine particles, TiO2 particles and ZnO particles. When present, quaternary ammonium salts may be a mass fraction of between about 1% to 30% of the mass of the antibacterial resin, preferably about 7.5% to about 15% of the mass of the antibacterial resin. When present, silver-containing nanoparticles may be a mass fraction of between about 0.01% and about 20% of the mass of the antibacterial resin, preferably 0.08% to 10% of the mass of the antibacterial resin.

In a third embodiment, the present invention is directed to dental composites comprising an antibacterial resin of the present invention and a filler. The filler is one or more of a glass filler, a ceramic filler, a polymer-based filler, and nanoparticles of amorphous calcium phosphate (NACP). In aspects where the filler is a glass filler, the glass filler may be barium boroaluminosilicate, strontium-alumino-fluoro-silicate glass, silicon dioxide, fluoroaluminosilicate glass, a ytterbium tri-fluoride filler, or a fiber glass filler. In certain aspects, the filler is barium boroaluminosilicate. In aspects where the filler is a ceramic filler, the ceramic filler may be a porcelain filler, a quartz filler, or a zirconia filler.

The dental composites may comprise one or more of the antibacterial resins of the present invention and one or more fillers. The amount of the one or more of the antibacterial resins in the dental composite is between about 1% to about 70% of the mass of the composite. In certain aspects, the antibacterial resin is a mass fraction of from about 10% to about 45%, about 20% to about 40%, or about 25% to about 35% of the dental composite. In certain other aspects, the antibacterial resin is a mass fraction of about 10%, about 15%, about 20%, about 25%, about 30%, or about 35% of the dental composite.

The amount of the one or more fillers in the dental composite is between about 5% to about 90% of the mass of the composite. In certain aspects, the filler is a mass fraction of from about 10% to about 85%, about 20% to about 85%, or about 30% to about 80% of the dental composite. In certain other aspects, the filler is a mass fraction of about 80%, about 75%, about 70%, about 65%, about 60%, about 55%, or about 50% of the composite.

When NACP is present as a filler, whether alone or in combination with one or more other fillers, it may make up between about 1% and 80% of the mass of the composite. In certain aspects, the NACP is a mass fraction of from about 5% to about 60%, about 10% to about 40%, about 15% to about 35%, or about 20% to about 30% of the composite. In certain other aspects, the NACP is a mass fraction of about 25%, about 25.5%, about 26%, about 26.5%, about 27%, about 27.5%, about 28%, about 28.5%, about 29%, about 29.5% or about 30% of the composite.

The NACP particles may range in size from about 10 nm to about 500 nm. In certain aspects, the NACP particles range in size from about 50 nm to about 300 nm, about 50 nm to about 200 nm, or about 75 nm to about 200 nm.

As indicated, in some aspects, the filler is two different fillers, for example, a glass filler and NACP, or a ceramic filler and NACP. In such aspects, the total amount of the two fillers in the dental composite will again be about 5% to about 90% of the mass of the composite. As a non-limiting example, the first filler (e.g., a glass filler) may be about 50% of the mass of the composite while the second filler (e.g., NACP) is about 20% of the mass of the composite. In another example, the first filler (e.g., a glass filler) is about 40% of the mass of the composite while the second filler (e.g., NACP) is about 30% of the mass of the composite. In a further example, the first filler (e.g., a glass filler) is about 35% of the mass of the composite while the second filler (e.g., NACP) is about 35% of the mass of the composite.

In a first specific embodiment, the present invention is directed to a dental composite comprising about 50% glass filler, about 20% NACP and about 30% antibacterial resin by mass of the composite, wherein the antibacterial resin comprises about 2.5% by mass DMADDM, DMAPDM, or DMAHDM as the antibacterial monomer.

In a second specific embodiment, the present invention is directed to a dental composite comprising about 50% glass filler, about 20% NACP and about 30% antibacterial resin by mass of the composite, wherein the antibacterial resin comprises about 5% by mass DMADDM, DMAPDM, or DMAHDM as the antibacterial monomer.

In a third specific embodiment, the present invention is directed to a dental composite comprising about 50% glass filler, about 20% NACP and about 30% antibacterial resin by mass of the composite, wherein the antibacterial resin comprises about 7.5% by mass DMADDM, DMAPDM, or DMAHDM as the antibacterial monomer.

In a fourth specific embodiment, the present invention is directed to a dental composite comprising about 50% glass filler, about 20% NACP and about 30% antibacterial resin by mass of the composite, wherein the antibacterial resin comprises about 10% by mass DMADDM, DMAPDM, or DMAHDM as the antibacterial monomer.

In certain aspects of each of these specific embodiments, the glass filler is barium boroaluminosilicate, the resin is a 1:1 mass ratio of bis-GMA and TEGDMA, and the NACP particles range in size from about 50 nm to about 200 nm.

### BRIEF DESCRIPTION OF THE DRAWINGS

FIGS. 1A-B—Increasing the monomer concentration decreased the fibroblast viability (n=6). At each concentration, the new antibacterial monomer with a chain length of 12 (DMADDM) had the highest fibroblast viability (p<0.05). DMADDM and MDPB are less cytotoxic, with higher cell viability, than BisGMA, a monomer commonly used in dental resins.

FIG. 2—A modified Menschutkin reaction was used to synthesize new antibacterial monomers: (A) DMAHM, and (B) DMADDM. DMAH=N,N-dimethylaminohexane. BEMA=2-bromoethyl methacrylate. DMAHM=dimethylaminohexane methacrylate. DMAD=1-(dimethylamino)docecane. DMADDM=dimethylaminododecyl methacrylate. EtOH=anhydrous ethanol. The number of the alkyl chain length units was 6 for DMAHM and 12 for DMADDM.

FIGS. 3A-B—FTIR spectra of reactants and products for: (A) DMAHM, and (B) DMADDM.

FIGS. 4A-B—Mechanical properties of composites: (A) flexural strength, and (B) elastic modulus (mean±sd; n=6). NACP+ODMADDM refers to NACP nanocomposite containing 0% DMADDM; NACP+0.75DMADDM refers to NACP nanocomposite containing 0.75% of DMADDM; and so on. Adding up to 3% of DMADDM into NACP nanocomposite resulted in no significant decrease in strength and elastic modulus. Horizontal line indicates values that are not significantly different from each other (p>0.1).

FIGS. 5A-E—Typical confocal laser scanning microscopy (CLSM) images of live/dead stained biofilms on composites. Live bacteria were stained green, and dead bacteria were stained red. Live/dead bacteria that were close to, or on the top of, each other produced yellow/orange colors. Composite control and NACP nanocomposite had primarily live bacteria. The DMADDM-containing nanocomposites showed substantial antibacterial activity.

FIGS. 6A-B—Dental plaque microcosm biofilms adherent on composites: (A) MTT metabolic activity, and (B) lactic acid production (mean±sd; n=6). NACP+ODMADDM refers to NACP nanocomposite containing 0% DMADDM; NACP+0.75DMADDM refers to NACP nanocomposite containing 0.75% of DMADDM; and so on. In each plot, values with dissimilar letters are significantly different (p<0.05).

FIGS. 7A-C—Colony-forming unit (CFU) counts for: (A) Total microorganisms, (B) total streptococci, and (C) mutans streptococci (mean±sd; n=6). NACP+ODMADDM refers to NACP nanocomposite containing 0% DMADDM; NACP+0.75DMADDM refers to NACP nanocomposite containing 0.75% of DMADDM; and so on. In each plot, values with dissimilar letters are significantly different (p<0.05). Note the log scale for the y-axis.

### DETAILED DESCRIPTION

Described herein are new antibacterial monomers that differ based on the length of the alkyl chain. The monomers include the following: dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). These antibacterial monomers are well suited for use in antibacterial resins and dental composites that are used in dental applications. Thus, the additional aspects of the present invention are directed to such resins and composites.

The antibacterial resins of the present invention comprise any resin (or combination of resins) that is suitable for dental use in a subject, such as a human, and one or more of the antibacterial monomers. The novel antibacterial monomers that are used in the preparation of the antibacterial resins greatly increase the ability of the resins to resist bacterial colonization and biofilm formation. Suitable resins will be those resins commonly used in dental applications. Such resins typically comprise a matrix that is of a hardenable dental polymer. Exemplary resins include bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer and a heat-cured polymer, as well combinations of two or more of these polymers. A suitable combination is Bis-GMA and TEGDMA at 1:1 mass ratio.

The resins used in the antibacterial resins of the invention may be rendered light-curable through the addition of appropriate compounds to the resin. For example, camphorquinone and ethyl 4-N,N-dimethylaminobenzoate may be added to a resin comprising Bis-GMA and TEGDMA thereby rendering the resin light-curable. In a particular embodiment, about 0.2% camphorquinone and about 0.8% ethyl 4-N,N-dimethylaminobenzoate may be added to a resin comprising Bis-GMA and TEGDMA in about a 1:1 mass ratio to render the resin light-curable.

The amount of the one or more antibacterial monomers present in the antibacterial resins of the present invention is a combined amount of antibacterial monomer of from about 0.5% to about 50% by mass of the antibacterial resin. In certain aspects, the antibacterial resin comprises a combined amount of from about 1% to about 25%, from about 2.5% to about 25%, from about 2.5% to about 20%, from about 2.5% to about 15%, from about 5% to about 25%, from about 5% to about 20%, from about 5% to about 15%, from about 7.5% to about 25%, from about 7.5% to about 20%, about 7.5% to about 15% or from about 7.5% to about 12.5% of the mass of the antibacterial resin. In particular aspects, the amount of the one or more antibacterial monomers present in the antibacterial resin is a combined amount of about 1, 2, 2.5, 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, 30, 31, 32, 33, 34, 35, 36, 37, 38, 39, 40, 41, 42, 43, 44, 45, 46, 47, 48, 49 or 50% of the mass of the antibacterial resin.

The antibacterial resins of the present invention may further comprise one or more additional antibacterial agents, including, but not limited to quaternary ammonium salts (QAS), silver-containing nanoparticles (NAg), chlorhexidine particles, TiO2 particles and ZnO particles. Suitable QASs include both polymerizable monomers and non-polymerizable small molecules, and include, but are not limited to, bis(2-methacryloyloxy-ethyl) dimethyl-ammonium bromide (QADM), methacryloyloxydodecylpyridinium bromide, methacryloxylethyl benzyl dimethyl ammonium chloride, methacryloxylethyl m-chloro benzyl dimethyl ammonium chloride, methacryloxylethyl cetyl dimethyl ammonium chloride, cetylpyridinium chloride, and methacryloxylethyl cetyl ammonium chloride, QAS chlorides, QAS bromides, QAS monomethacrylates, QAS dimethacrylates, and pre-fabricated QAS particles.

Suitable silver-containing nanoparticles include, but are not limited to, silver 2-ethylhexanoate salt, silver-containing glass particles and silver benzoate. In addition to silver salts, pre-formed silver nanoparticles can be used.

When present, the QAS may make up between about 1% and about 30% of a mass fraction of the antibacterial resin. In certain aspects, the QAS will make up between about 2% and about 25%, about 5% and about 20%, or about 7.5% and about 15% of a mass fraction of the antibacterial resin, or about 1%, 2.5%, 5%, 7.5%, 10%, 12.5, 15%, 17.5%, 20%, 22.5%, 25%, 27.5% or 30% of a mass fraction of the antibacterial resin.

When present, NAg may make up between about 0.01% and about 20% of a mass fraction of the antibacterial resin. In certain aspects, NAg will make up between about 0.05% and about 5%, or 0.08% and about 10%, of a mass fraction of the antibacterial resin, or about 0.01%, 0.08%, 0.15%, 0.20%, 0.25%, 0.30%, 0.35%, 0.40%, 0.45%, 0.50%, 1.0%, 1.5%, 2.0%, 2.5%, 3.0%, 3.5%, 4.0%, 4.5% or 5.0% of a mass fraction of the antibacterial resin. In one aspect, NAg makes up about 0.08% of a mass fraction of the antibacterial resin. The silver particle size can range from about 1 nm to about 1000 nm, and in one aspect, from about 2 nm to about 500 nm.

The dental composites of the present invention comprise an antibacterial resin of the present invention and a filler. The dental composites described herein: (1) are mechanically stronger than a resin-modified glass ionomer; (2) neutralize cariogenic acid and raise the acidic pH to a safe level; and (3) possess antibacterial properties against cariogenic bacteria such as S. mutans.

The antibacterial resins used in the dental composites may be any of the antibacterial resins described herein, or a combination of the antibacterial resins of the present invention. The amount of antibacterial resin present in the dental composites of the present invention may also vary, but the antibacterial resin will generally comprise about 1% to about 70% of the mass of the composite. In certain aspects, the antibacterial resin will comprise about 1% to about 45%, about 1% to about 40%, about 1% to about 35%, about 1% to about 30%, about 1% to about 25%, about 1% to about 20%, about 1% to about 15%, about 2.5% to about 45%, about 2.5% to about 40%, about 2.5% to about 35%, about 2.5% to about 30%, about 2.5% to about 25%, about 2.5% to about 20%, about 2.5% to about 15%, about 5% to about 45%, about 5% to about 40%, about 5% to about 35%, about 5% to about 30%, about 5% to about 25%, about 5% to about 20%, about 5% to about 15%, about 7.5% to about 45%, about 7.5% to about 40%, about 7.5% to about 35%, about 7.5% to about 30%, about 7.5% to about 25%, about 7.5% to about 20%, about 7.5% to about 15%, about 10% to about 45%, about 10% to about 40%, about 10% to about 35%, about 10% to about 30%, about 10% to about 25%, about 10% to about 20%, about 10% to about 15%, about 15% to about 45%, about 15% to about 40%, about 15% to about 35%, about 15% to about 30%, about 15% to about 25%, about 15% to about 20%, about 20% to about 45%, about 20% to about 40%, about 20% to about 35%, about 20% to about 30%, about 20% to about 25%, about 25% to about 45%, about 25% to about 40%, about 25% to about 35%, or about 25% to about 30%. In certain other aspects, the antibacterial resin is a mass fraction of from about 10%, about 15%, about 20%, about 25%, about 30%, or about 35% of the dental composite. In particular aspects, the antibacterial resin will comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70% of the mass of the composite.

The filler is used to increase the strength of the composite. Suitable fillers include glass fillers, ceramic fillers, polymer-based fillers and NACP, or any combination thereof. Particular examples of suitable glass fillers include barium boroaluminosilicate, strontium-alumino-fluoro-silicate glass, silicon dioxide, fluoroaluminosilicate glass, a ytterbium tri-fluoride filler, and a fiber glass filler. Particular examples of suitable ceramic fillers include any dental ceramic such as a porcelain filler, a quartz filler, and a zirconia filler. Polymer-based filler includes dental polymer that is pre-polymerized and then ground into filler particles, and polymer fibers. NACP comprises nanometer-sized amorphous calcium phosphate (Ca₃[PO₄]₂) particles that can be used to produce photo-cured nanocomposites with high Ca and PO₄ release, improved mechanical properties, and improved antibacterial properties. The dental composites that include NACP exhibit greatly increased ion release at acidic, cariogenic pH, when these ions are most needed to inhibit caries.

The amount of filler present in the dental composites of the present invention may vary, but the filler will generally comprise about 5% to about 90% of the mass of the composite. In certain aspects, the filler will comprise about 10% to about 85%, about 10% to about 80%, about 10% to about 70%, about 10% to about 60%, about 10% to about 50%, about 10% to about 40%, about 20% to about 80%, about 20% to about 70%, about 20% to about 60%, about 20% to about 50%, about 30% to about 80%, about 30% to about 70%, about 30% to about 60%, about 40% to about 80%, about 40% to about 70%, about 40% to about 60%, about 50% to about 80%, about 50% to about 70%, or about 45% to about 55% of the mass of the composite. In certain other aspects, the filler is a mass fraction of about 80%, about 75%, about 70%, about 65%, about 60%, about 55%, or about 50% of the composite. In particular aspects, the filler comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84 or 85% of the mass of the composite.

When present, the amount of NACP included as a filler may vary, but the NACP will generally comprise about 1% to about 80% of the mass of the composite. In certain aspects, the NACP will comprise about 5% to about 70%, about 5% to about 60%, about 5% to about 50%, about 5% to about 40%, about 10% to about 90%, about 10% to about 80%, about 10% to about 70%, about 10% to about 60%, about 10% to about 50%, about 10% to about 40%, about 15% to about 70%, about 15% to about 60%, about 15% to about 50%, about 15% to about 40%, about 20% to about 90%, about 85% to about 70%, about 20% to about 80%, about 20% to about 70%, about 20% to about 60%, about 20% to about 50%, about 20% to about 40%, about 25% to about 70%, about 25% to about 60%, about 25% to about 50%, about 25% to about 40%, about 30% to about 90%, about 30% to about 85%, about 30% to about 80%, about 30% to about 75%, about 30% to about 70%, about 30% to about 60%, about 15% to about 45%, about 15% to about 35%, or about 15% to about 25% of the mass of the composite. In certain other aspects, the NACP is a mass fraction of about 25%, about 25.5%, about 26%, about 26.5%, about 27%, about 27.5%, about 28%, about 28.5%, about 29%, about 29.5% or about 30% of the composite. In particular aspects, the NACP will comprise about 5, 6, 7, 8, 9, 10, 11, 12, 13, 14, 15, 16, 17, 18, 19, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90% of the mass of the composite.

The size of the particles of the filler will depend on the identity of the filler or fillers. As an example, in an embodiment where barium boroaluminosilicate glass particles serve as the filler or one of fillers, the median particle diameter may be between about 0.1 and about 10 or between about 1.0 μm and about 5 μm. Thus, the median particle diameter of the fillers used in the composites of the present invention may, in one aspect where barium boroaluminosilicate glass particles is the filler or one of fillers, be between about 0.1 and about 10 or between about 1.0 μm and about 5 μm. In certain aspects, where barium boroaluminosilicate glass particles is the filler or one of fillers, the median particle diameter may be about 0.6 μm, 0.8 μm, 1.0 μm, 1.2 μm, 1.4 μm, 1.6 μm, 1.8 and 2.0 μm. The skilled artisan will understand that the particle size of the particular filler used will depend on the identity of the filler or fillers, and while the sizes provided here are with respect to barium boroaluminosilicate glass particles, similar sizes may pertain to one or more of the alternative fillers described herein.

When NACP is included as a filler, it will vary in size, but at least about 50, 55, 60, 65, 70, 75, 80, 85, 86, 87, 88, 89, 90, 91, 92, 93, 94, 95, 96, 97, 98 or 99% of the particles, or all of the particles (100%), have an average diameter of between about 10 nm and about 500 nm. In certain aspects, the average diameter will be between about 25 nm and about 400 nm, about 50 nm and about 300 nm, about 75 nm and about 200 nm, or about 100 nm and about 150 nm. In a particular aspect, the NACP particles have an average diameter of between about 50 nm and about 200 nm.

Depending on the identity of the filler, the particles comprising the filler may be silanized. Suitable means for silanization are known to the skilled artisan and include, but are not limited to, a mixture of about 4% 3-methacryloxypropyltrimethoxysilane and about 2% n-propylamine. In one embodiment, the filler comprises barium boroaluminosilicate glass particles, where the particles are silanized. In another embodiment, the filler comprises silanized barium boroaluminosilicate glass particles having a median particle diameter of about 1.4 μm.

In certain aspects, the dental composites of the present invention will comprise about 20% antibacterial resin and about 80% filler by mass of the composite. In other aspects, the dental composites of the present invention will comprise about 21% antibacterial resin and about 79% filler, 22% antibacterial resin and about 78% filler, 23% antibacterial resin and about 77% filler, 24% antibacterial resin and about 76% filler, 25% antibacterial resin and about 75% filler, 26% antibacterial resin and about 74% filler, 27% antibacterial resin and about 73% filler, 28% antibacterial resin and about 72% filler, 29% antibacterial resin and about 71% filler, 30% antibacterial resin and about 70% filler, 31% antibacterial resin and about 69% filler, 32% antibacterial resin and about 68% filler, 33% antibacterial resin and about 67% filler, 34% antibacterial resin and about 66% filler, 35% antibacterial resin and about 65% filler, 36% antibacterial resin and about 64% filler, 37% antibacterial resin and about 63% filler, 38% antibacterial resin and about 62% filler, 39% antibacterial resin and about 61% filler, or 40% antibacterial resin and about 60% filler by mass of the composite.

In non-limiting examples of the present invention, the dental composite comprises:

(i) about 70% by mass filler and about 30% by mass antibacterial resin, wherein the antibacterial resin comprises about 2.5% antibacterial monomer by mass;
(ii) about 70% by mass filler and about 30% by mass antibacterial resin, wherein the antibacterial resin comprises about 5% antibacterial monomer by mass;
(iii) about 70% by mass filler and about 30% by mass antibacterial resin, wherein the antibacterial resin comprises about 7.5% antibacterial monomer by mass; and
(iv) about 70% by mass filler and about 30% by mass antibacterial resin, wherein the antibacterial resin comprises about 10% antibacterial monomer by mass.

In additional non-limiting examples of the present invention, the dental composite comprises:

(i) about 50% glass filler, about 20% NACP and about 30% antibacterial resin by mass of the composite, wherein the antibacterial resin comprises about 2.5% by mass antibacterial monomer;
(ii) about 50% glass filler, about 20% NACP and about 30% antibacterial resin by mass of the composite, wherein the antibacterial resin comprises about 5% by mass antibacterial monomer;
(iii) about 50% glass filler, about 20% NACP and about 30% antibacterial resin by mass of the composite, wherein the antibacterial resin comprises about 7.5% by mass antibacterial monomer; and
(iv) about 50% glass filler, about 20% NACP and about 30% antibacterial resin by mass of the composite, wherein the antibacterial resin comprises about 10% by mass antibacterial monomer.

In further non-limiting examples of the present invention, the dental composite comprises:

(i) about 70% barium boroaluminosilicate glass filler and about 30% BisGMA-TEGDMA (1:1 mass ratio) antibacterial resin by mass of the composite, wherein the antibacterial resin comprises about 2.5% by mass DMADDM, DMAPDM, or DMAHDM as the antibacterial monomer;
(ii) about 70% barium boroaluminosilicate glass filler and about 30% BisGMA-TEGDMA (1:1 mass ratio) antibacterial resin by mass of the composite, wherein the antibacterial resin comprises about 5% by mass DMADDM, DMAPDM, or DMAHDM as the antibacterial monomer;
(iii) about 70% barium boroaluminosilicate glass filler and about 30% BisGMA-TEGDMA (1:1 mass ratio) antibacterial resin by mass of the composite, wherein the antibacterial resin comprises about 7.5% by mass DMADDM, DMAPDM, or DMAHDM as the antibacterial monomer; and
(iv) about 70% barium boroaluminosilicate glass filler and about 30% BisGMA-TEGDMA (1:1 mass ratio) antibacterial resin by mass of the composite, wherein the antibacterial resin comprises about 10% by mass DMADDM, DMAPDM, or DMAHDM as the antibacterial monomer.

In still further non-limiting examples of the present invention, the dental composite comprises:

(i) about 50% barium boroaluminosilicate glass filler, about 20% NACP and about 30% BisGMA-TEGDMA (1:1 mass ratio) antibacterial resin by mass of the composite, wherein the antibacterial resin comprises about 2.5% by mass DMADDM, DMAPDM, or DMAHDM as the antibacterial monomer;
(ii) about 50% barium boroaluminosilicate glass filler, about 20% NACP and about 30% BisGMA-TEGDMA (1:1 mass ratio) antibacterial resin by mass of the composite, wherein the antibacterial resin comprises about 5.0% by mass DMADDM, DMAPDM, or DMAHDM as the antibacterial monomer;
(iii) about 50% barium boroaluminosilicate glass filler, about 20% NACP and about 30% BisGMA-TEGDMA (1:1 mass ratio) antibacterial resin by mass of the composite, wherein the antibacterial resin comprises about 7.5% by mass DMADDM, DMAPDM, or DMAHDM as the antibacterial monomer; and
(iv) about 50% barium boroaluminosilicate glass filler, about 20% NACP and about 30% BisGMA-TEGDMA (1:1 mass ratio) antibacterial resin by mass of the composite, wherein the antibacterial resin comprises about 10% by mass DMADDM, DMAPDM, or DMAHDM as the antibacterial monomer.

The dental composites, antibacterial resins and antibacterial monomers of the present invention are suitable for use in the teeth of mammals, including primates such as human or non-human primates, and those of dogs, cats, horses, cattle, pigs, goats and sheep, for example.

The dental composites described herein can be used in a method of inhibiting growth of aciduric bacteria on a surface of a tooth of a subject, comprising restoring a surface of the tooth from which a decayed portion has been removed by applying a dental composite as described herein to the surface of the tooth, thereby inhibiting growth of aciduric bacteria on the tooth of the subject.

The dental composites described herein can also be used in a method of inhibiting further decay of a decaying tooth in a subject, comprising restoring a surface of the tooth from which a decayed portion has been removed by applying a dental composite as described herein to the surface of the tooth, thereby inhibiting further decay of the decaying tooth in the subject.

### EXAMPLES

### Example 1

### Synthesis of Quaternary Ammonium Methacrylates with Different Alkyl Chain Lengths

Synthesis of a variety of quaternary ammonium salt (QAS) monomethacrylates was carried out using a Menschutkin reaction (Menschutkin et al. 1890; Antonucci et al. 2012). The reaction proceeds by the addition reaction of tertiary amines with organo-halides. To form a QAS with a reactive methacrylate groups, 2-(dimethylamino)ethyl methacrylate (DMAEMA) was chosen as methacrylate-containing tertiary amine. In order to investigate the effect of chain length from the quaternary ammonium site on the antibacterial properties of QAS monomers, seven different alkyl organo-halides were each chosen to react with BEMA.

Examples are summarized in Table 1. Other chain lengths including 2, 4, 5, 7, 8, 10, 11, 13, 14, 16, 17, 19, 20, 21, 23, 24, and 25 can be similarly synthesized.

TABLE 1
Summary of the Reaction of Dimethylamino methacrylate
(DMAEMA) with Various Organo-Halides

| | | | Alkyl |
|---|---|---|---|
| | | | Chain |
| Tertiary Amine | Alkyl Organo-Halide | Product | Length |
| Dimethylamino | 1-bromopropane (BP) | DMAPM | 3 |
| methacrylate | 1-bromohexane (BH) | DMAHM | 6 |
| (DMAEMA) | 1-bromoheptane (BHP) | DMAHPM | 7 |
| | 1-bromooctane (BO) | DMAOM | 8 |
| | 1-bromononane (BN) | DMANM | 9 |
| | 1-bromodecane (BD) | DMADM | 10 |
| | 1-bromoundecane (BUD) | DMAUDM | 11 |
| | 1-bromododecane (BDD) | DMADDM | 12 |
| | 1-bromotridecane (BTD) | DMATDM | 13 |
| | 1-bromotetradecane (BTTD) | DMATTDM | 14 |
| | 1-bromopentadecane (BPD) | DMAPDM | 15 |
| | 1-bromohexadecane (BHD) | DMAHDM | 16 |
| | 1-bromoheptadecane (BHPD) | DMAHPDM | 17 |
| | 1-bromooctadecane (BOD) | DMAODM | 18 |
| | 1-bromononadecane(BND) | DMANDM | 19 |
| | 1-bromoicosane (BIO) | DMAIOM | 20 |
| | 1-bromohenicosane (BHO) | DMAHOM | 21 |
| | 1-bromodocosane (BDO) | DMADOM | 22 |

Synthesis of QAS with Chain Length=3

In a 20 mL scintillation vial, 10 mmol of 2-(dimethylamino)ethyl methacrylate (DMAEMA, Sigma Aldrich, St. Louis Mo.) and 10 mmol of 1-bromopropane (BP, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=6

In a 20 mL scintillation vial, 10 mmol of DMAEMA and 10 mmol of 1-bromohexane (BH, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=9

In a 20 mL scintillation vial, 10 mmol of DMAEMA and 10 mmol of 1-bromononane (BN, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=12

In a 20 mL scintillation vial, 10 mmol of DMAEMA and 10 mmol of 1-bromododecane (BDD, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=15

In a 20 mL scintillation vial, 10 mmol of DMAEMA and 10 mmol of 1-bromopentadecane (BPD, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=18

In a 20 mL scintillation vial, 10 mmol of DMAEMA and 10 mmol of 1-bromooctadecane (BOD, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

Synthesis of QAS with Chain Length=22

In a 20 mL scintillation vial, 10 mmol of DMAEMA and 10 mmol of 1-bromodocosane (BDO, TCI America, Portland Oreg.) were added. To this mixture, 3 g of ethanol was added as a solvent. A magnetic stir bar was added, and the vial was capped and stirred at 70° C. for 24 h. After the reaction was complete, the ethanol solvent was removed via evaporation at room temperature over several days.

### Characterization of Reaction Products

FTIR spectra (Nicolet 6700, Thermo Scientific, Waltham, Mass.) of the starting materials and the viscous products were collected between two KBr windows in the 4000 cm⁻¹ to 400 cm⁻¹ region with 128 scans at 4 cm⁻¹ resolution. Water and CO₂ bands were removed from all spectra by subtraction. ¹H NMR spectra (GSX 270, JEOL USA Inc., Peabody, Mass.) of the starting materials and products were taken in deuterated chloroform at a concentration of approximately 3%. All spectra were run at room temperature, 15 Hz sample spinning, 45° tip angle for the observation pulse, and a 10 s recycle delay, for 64 scans.

### Cytotoxicity of Monomers via MTT Assay

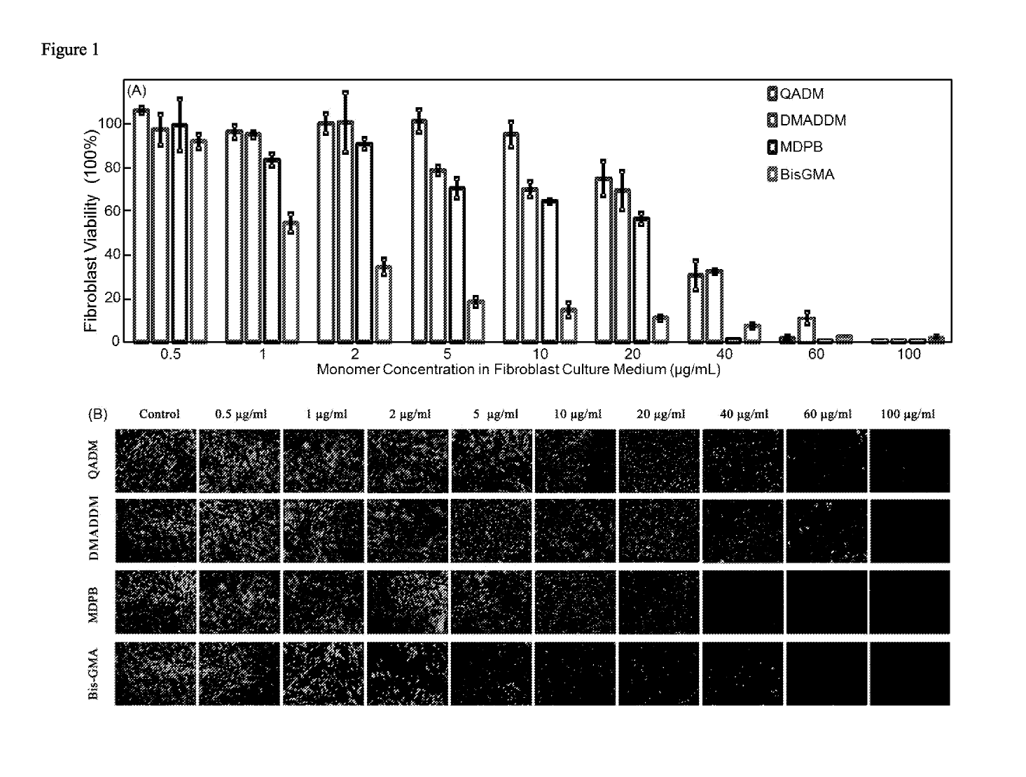

Human gingival fibroblasts (HGF, ScienCell) were cultured in a fibroblast medium (FM). Each unpolymerized monomer was dissolved in FM, at concentrations of: 0 (control), 0.5, 1, 2, 5, 10, 20, 40, 60, and 100 μg/mL (Huang L et al. 2011; Chai Z et al. 2011). Then, HGF were seeded in 96-well plates at 5,000 cells per well. After 2 d, 20 μL of MTT solution was added (Chai Z et al. 2011). After 4 h, the unreacted dye was removed and 150 μL, of dimethyl sulfoxide was added. Absorbance was measured via the microplate reader at 492 nm. Relative fibroblast viability=absorbance of monomer sample/absorbance of control without monomer (Chai Z et al. 2011). The results are provided in FIG. 1A. Cells were also live/dead stained (Molecular Probes) and examined with fluorescence microscopy (TE2000-S, Nikon) as shown in FIG. 1B.

Incorporating antibacterial monomers with different chain lengths into dental composites

The composite matrix is a resin or combinations of resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate) and PMGDM (pyromellitic acid glycerol dimethacrylate). The composite fillers may include calcium phosphate nanoparticles such as nanoparticles of amorphous calcium phosphate (NACP). The NACP particles range in size from about 10 nm to about 500 nm. The NACP filler level ranges from about 5% to about 90% of the mass of the composite. The composite can contain other fillers such as usual dental glass fillers. Alternatively, the composite may contain glass fillers, without calcium phosphate fillers, in which the incorporation of the new antibacterial monomers will render the composite strongly antibacterial. The new antibacterial composite may contain fibers and whiskers as mechanical reinforcement.

One or more antibacterial monomers with various chain lengths can be incorporated into the composite, at antibacterial resin mass fractions ranging from 1% to 50% of the composite, preferably 2% to 20% of the composite. Other techniques for producing the dental composites are disclosed in WO 2012/003290, incorporated herein by reference in its entirety.

### Data on Longer Chain Length

A new antibacterial monomer with a chain length of 16 was also prepared which exhibited a MBC=0.61 μg/mL and a MIC=0.305 μg/mL. These values are an order of magnitude more potent than those for chain length 12 reported below.

### Example 2

### Alternative Means for Synthesis of Quaternary Ammonium Methacrylates (QAMs)

A modified Menschutkin reaction approach was used to synthesize the new QAMs. This method uses a tertiary amine group to react with an organo-halide, as described in previous studies (Antonucci J M et al. 2012; Cheng L et al. 2012a). A benefit of this reaction is that the reaction products are generated at virtually quantitative amounts and require minimal purification (Antonucci J M et al. 2012). In the present study, 2-bromoethyl methacrylate (BEMA) was the organo halide. N,N-dimethylaminohexane (DMAH) and 1-(dimethylamino) docecane (DMAD) were the two tertiary amines.

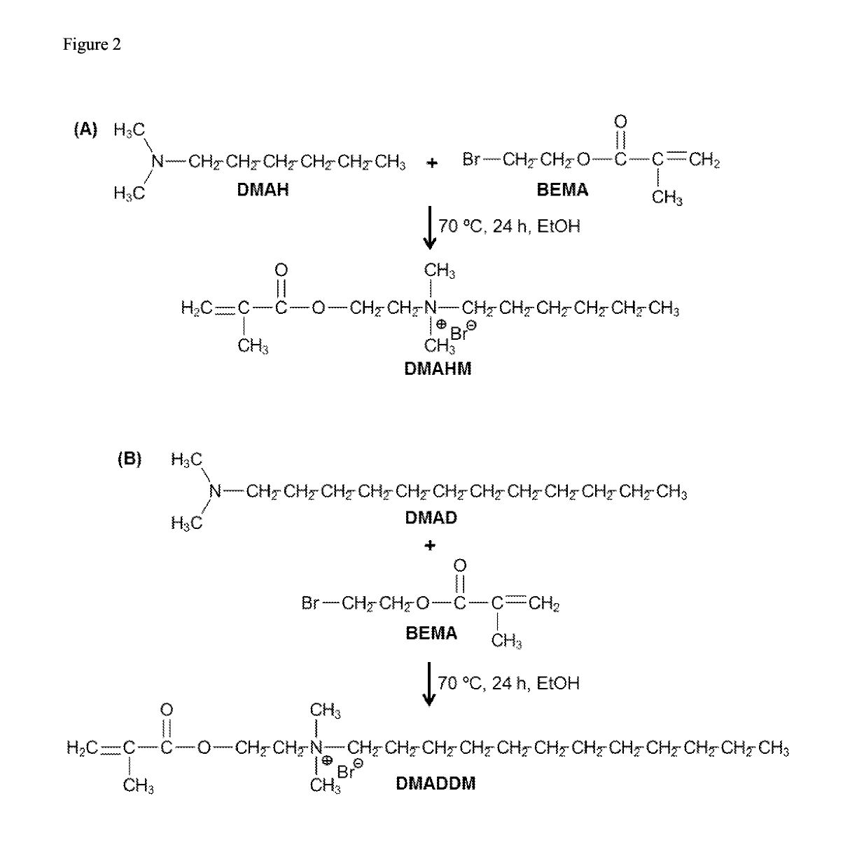

The scheme of synthesis of dimethylaminohexane methacrylate (DMAHM) is shown in FIG. 2A. Ten mmol of DMAH (Tokyo Chemical Industry, Tokyo, Japan), 10 mmol of BEMA (Monomer-Polymer and Dajac Labs, Trevose, Pa.), and 3 g of ethanol were added to a 20 mL scintillation vial with a magnetic stir bar. The vial was capped and stirred at 70° C. for 24 h. After the reaction was complete, the ethanol solvent was removed via evaporation at room temperature over several days. This yielded DMAHM as a clear liquid.

The scheme of synthesis of dimethylaminododecyl methacrylate (DMADDM) is shown in FIG. 2B. In a 20 mL scintillation vials were added 10 mmol of DMAD (Tokyo Chemical Industry), 10 mmol of BEMA, and 3 g of ethanol. A magnetic stir bar was added, and the vial was capped and stirred at 70° C. for 24 h. After the reaction was complete, the solvent was removed via evaporation. The number of the alkyl chain length units was 6 for DMAHM and 12 for DMADDM (FIG. 2).

To characterize the reaction products, Fourier transform infrared spectroscopy (FTIR, Nicolet 6700, Thermo Scientific, Waltham, Mass.) was used. FTIR spectra of the starting materials and the viscous products were collected between two KBr windows in the 4000 cm⁻¹ to 400 cm⁻¹ region with 128 scans at 4 cm⁻¹ resolution (Antonucci J M et al. 2012). Water and CO₂ bands were removed from all spectra by subtraction. ¹H NMR spectra (GSX 270, JEOL, Peabody, Mass.) of the starting materials and products were taken in deuterated chloroform at a concentration of approximately 3%. All spectra were run at room temperature, 15 Hz sample spinning, 45° tip angle for the observation pulse, and a 10 s recycle delay, for 64 scans (Antonucci J M et al. 2012).

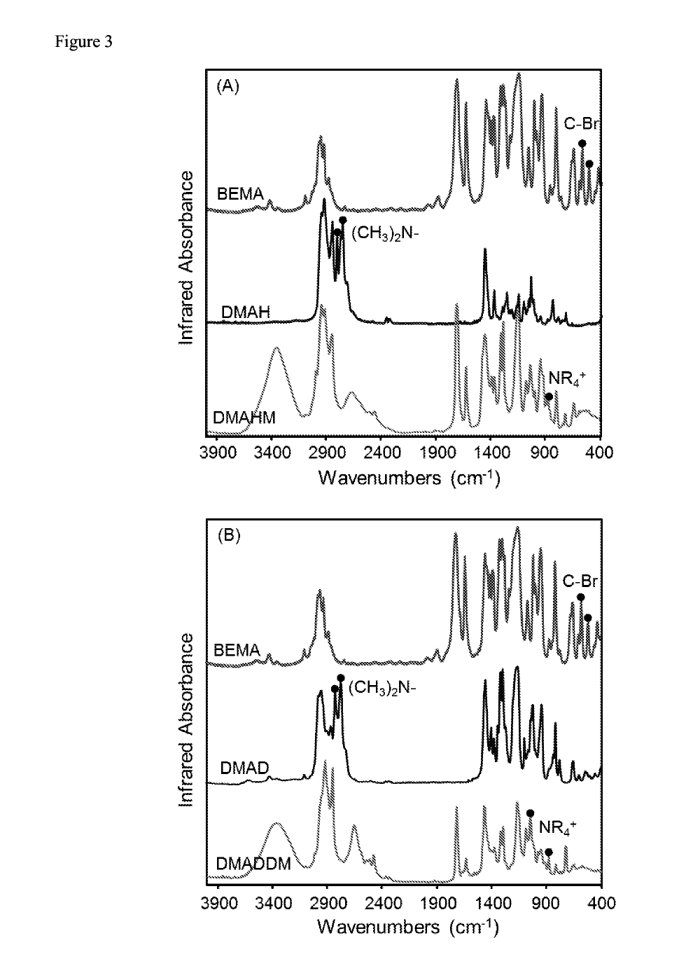

Representative results are provided in FIG. 3. The characterization using FTIR and ¹H NMR indicated that the Menschutkin reaction was successful. The infrared spectroscopy showed the disappearance of C—Br and tertiary amine groups, and the appearance of quaternary ammonium group that resulted from the reaction. In each plot, the appearance of the NR₄⁺ peak in the last curve corresponded to the formation of the quaternary ammonium group. In FIG. 3A, FTIR showed that the C—Br absorption bands from BEMA (575 cm⁻¹, 512 cm⁻¹) in curve 1 and the (CH₃)₂N⁻ bands (2822 cm⁻¹, 2771 cm⁻¹) from DMAH in curve 2 disappeared in curve 3. This indicated that the bromine group in BEMA successfully reacted with the amine group in DMHA to form the quaternary ammonium group. The appearance in curve 3 of the NR₄⁺ peak corresponded to the formation of the quaternary ammonium group and, hence, DMAHM was successfully synthesized. Similarly, FIG. 3B showed the synthesis of DMADDM from the reaction of BEMA and DMAD.

### Minimum Inhibitory Concentration (MIC) and Bactericidal Concentration (MBC)

MIC and MBC were measured using S. mutans (ATCC 700610, UA159, American Type Culture, Manassas, Va.). S. mutans is a cariogenic, aerotolerant anaerobic bacterium and the primary causative agent of dental caries (Loesche 1986). MIC and MBC were determined via serial microdilution assays (Imazato S et al. 2006; Huang L et al. 2011). Unpolymerized DMAHM or DMADM monomer was dissolved in brain heart infusion (BHI) broth (BD, Franklin Lakes, N.J.) to give a final concentration of 200 mg/mL. From these starting solutions, serial two fold dilutions were made into 1 mL volumes of BHI broth. 15 μL of stock S. mutans was added to 15 mL of BHI broth with 0.2% sucrose and incubated at 37° C. with 5% CO₂. Overnight cultures of S. mutans were adjusted to 2×10⁶ CFU/mL with BHI broth, and 50 μL of inocula was added to each well of a 96-well plate containing 50 μL of a series of antibacterial monomer dilution broths. BHI broth with 1×10⁶ CFU/mL bacteria suspension without antibacterial agent served as negative control. Chlorhexidine diacetate (CHX) (Sigma, St. Louis, Mo.) served as positive control. The previously-synthesized QADM (Antonucci J M et al. 2012; Cheng L et al. 2012a) served as an antibacterial monomer control. After incubation at 37° C. in 5% CO₂ for 48 h, the wells were read for turbidity, referenced by the negative and positive control wells. MIC was determined as the endpoint (the well with the lowest antibacterial agent concentration) where no turbidity could be detected with respect to the controls (Huang L et al. 2011). To determine MBC, an aliquot of 50 μL from each well without turbidity was inoculated on BHI agar plates and incubated at 37° C. in 5% CO₂ for 48 h. MBC was determined as the lowest concentration of antibacterial agent that produced no colonies on the plate. The tests were performed in triplicate (Huang L et al. 2011). The MIC and MBC values of the antibacterial agents against S. mutans are listed in Table 2.

TABLE 2
MIC and MBC values of various antibacterial
agents against S. mutans*

| | | |
|---|---|---|
| Compound | MBC | MIC |

| | | | | |
|---|---|---|---|---|
| QADM | 2.5 × 10⁴ | μg/mL | 1.25 × 10⁴ | μg/mL |
| DMAHM | 3.13 × 10³ | μg/mL | 1.56 × 10³ | μg/mL |
| DMADDM | 12.21 | μg/mL | 6.10 | μg/mL |
| CHX | 3.91 | μg/mL | 1.95 | μg/mL |

*CHX = Chlorhexidine. QADM = quaternary ammonium dimethacrylate. DMAHM = dimethylaminohexane methacrylate. DMADDM = dimethylaminododecyl methacrylate. Tests were repeated in triplicate.

A lower concentration of the antibacterial agent needed to inhibit the bacteria indicates a higher antibacterial potency. The new DMAHM with an alkyl chain length of 6 was more potent than the previously-synthesized QADM. In dramatic contrast, the new DMADDM with an alkyl chain length of 12 was much more strongly antibacterial than DMAHM. The MIC and MBC of DMADDM was more than two orders of magnitude lower than those of MDAHM, and approached those of the CHX control.

### Processing of DMADDM-NACP Nanocomposite

A spray-drying technique as described previously in Chow L C et al. (2004) was used to make NACP (Ca₃[PO₄]₂). Calcium carbonate (CaCO₃, Fisher, Fair Lawn, N.J.) and dicalcium phosphate anhydrous (CaHPO₄, Baker Chemical, Phillipsburg, N.J.) were dissolved into an acetic acid solution to obtain final Ca and P ionic concentrations of 8 mmol/L and 5.333 mmol/L, respectively. This resulted in a Ca/P molar ratio of 1.5, the same as that for ACP. This solution was sprayed into a heated chamber, and an electrostatic precipitator (AirQuality, Minneapolis, Minn.) was used to collect the dried particles. This method produced NACP with a mean particle size of 116 nm, as measured in a previous study (Xu H H K et al. 2011). Other techniques for producing NACP are disclosed in WO 2012/003290, incorporated herein by reference in its entirety.

Because DMADDM exhibited a much greater antibacterial potency than DMAHM and QADM, DMADDM was used for incorporation into the NACP nanocomposite to obtain antibacterial properties. BisGMA (bisphenol glycidyl dimethacrylate) and TEGDMA (triethylene glycol dimethacrylate) (Esstech, Essington, Pa.) were mixed at a mass ratio=1:1, and rendered light-curable with 0.2% camphorquinone and 0.8% ethyl 4-N,N-dimethylaminobenzoate (mass fractions). DMADDM was mixed with the photo-activated BisGMA-TEGDMA resin at the following DMADDM/(BisGMA-TEGDMA+DMADDM) mass fractions: 0%, 2.5%, 5%, 7.5% and 10%, yielding five groups of resin, respectively. A dental barium boroaluminosilicate glass of a median particle size of 1.4 μm (Caulk/Dentsply, Milford, DE) was silanized with 4% 3-methacryloxypropyltrimethoxysilane and 2% n-propylamine (Xu H H K et al. 2011). The NACP and glass particles were mixed into each resin, at the same filler level of 70% by mass, with 20% of NACP and 50% of glass (Xu H H K et al. 2011). Because the resin mass fraction was 30% in the composite, the five DMADDM mass fractions in the composite were 0%, 0.75%, 1.5%, 2.25% and 3%, respectively. Other techniques for producing the dental composites are disclosed in WO 2012/003290, incorporated herein by reference in its entirety.

Six composites were tested: Five NACP nanocomposites at the five DMADDM mass fractions described above, and a commercial control composite. Renamel (Cosmedent, Chicago, Ill.) served as a control composite. It consisted of nanofillers of 20-40 nm in size, at 60% filler level in a multifunctional methacrylate ester resin. For mechanical testing, each paste was placed into rectangular molds of 2×2×25 mm. For biofilm experiments, each paste was placed into disk molds of 9 mm in diameter and 2 mm in thickness. The specimens were photo-cured (Triad 2000, Dentsply, York, Pa.) for 1 min on each side. The specimens were then incubated in distilled water at 37° C. for 24 hours prior to mechanical or biofilm testing.

### Mechanical Testing

A computer-controlled Universal Testing Machine (5500R, MTS, Cary, N.C.) was used to fracture the specimens in three-point flexure using a span of 10 mm and a crosshead speed of 1 mm/min. Flexural strength S was measured as: S=3PₘₐₓL/(2bh²), where Pₘₐₓ is the load-at-failure, L is span, b is specimen width and h is specimen thickness. Elastic modulus E was measured as: E=(P/d)(L³/[4bh³]), where load P divided by displacement d is the slope in the linear elastic region of the load-displacement curve. The specimens were taken out of the water and fractured within several minutes while still being wet (Cheng L et al. 2012a).

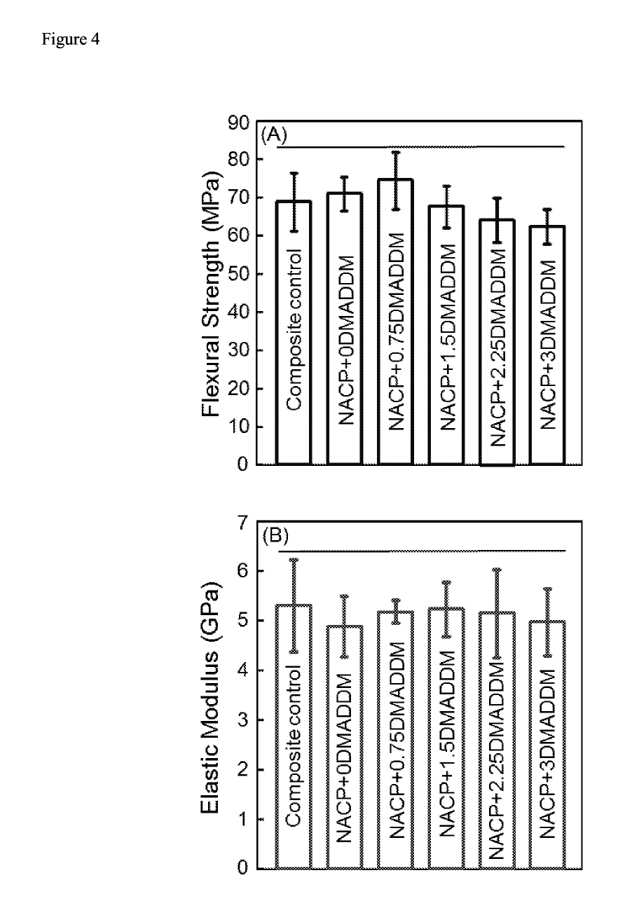

FIG. 4 plots (A) flexural strength, and (B) elastic modulus of the composites (mean±sd; n=6). The NACP nanocomposite with various DMADDM mass fractions had strengths similar to that of the commercial composite control, which was not antibacterial and had no Ca and P ion release (p>0.1). The elastic moduli of DMADDM-NACP nanocomposites were also similar to those of the NACP nanocomposite without DMADDM and the composite control (p>0.1).

### Dental Plaque Microcosm Biofilm and Live/Dead Assay

The dental plaque microcosm biofilm model used human saliva as inoculum. Saliva was collected from a healthy adult donor following a previous study (Cheng L et al. 2012b). The donor had natural dentition without active caries or periopathology, and without the use of antibiotics within the last 3 months. The donor did not brush teeth for 24 h and abstained from food or drink intake for at least 2 h prior to donating saliva (Cheng L et al. 2012b). Stimulated saliva was collected during parafilm chewing and kept on ice. The saliva was diluted in sterile glycerol to a concentration of 70% saliva and 30% glycerol (Cheng L et al. 2012b), and stored at −80° C.

The saliva-glycerol stock was added, with 1:50 final dilution, into the growth medium as inoculum. The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; haemin, 0.001 g/L; vitamin K₁, 0.0002 g/L, at pH 7 (McBain A J 2009). Composite disks were sterilized in ethylene oxide (Anprolene AN 74i, Andersen, Haw River, N.C.). 1.5 mL of inoculum was added to each well of 24-well plates with a composite disk, and incubated in 5% CO₂ at 37° C. for 8 h. The disks were then transferred to new 24-well plates filled with fresh medium and incubated. After 16 h, the disks were transferred to new 24-well plates with fresh medium and incubated for 24 h. This totaled 48 h of incubation, which was shown to be adequate to form dental plaque microcosm biofilms on resins (Cheng L et al. 2012b; Zhang K et al. 2012).

After 48 h of growth, the microcosm biofilms adherent on the disks were gently washed three times with phosphate buffered saline (PBS), and then stained using the BacLight live/dead bacterial viability kit (Molecular Probes, Eugene, Oreg.) (Cheng L et al. 2012b; Zhang K et al. 2012). Live bacteria were stained with Syto 9 to produce a green fluorescence, and bacteria with compromised membranes were stained with propidium iodide to produce a red fluorescence. The stained disks were examined using a confocal laser scanning microscopy (CLSM 510, Carl Zeiss, Thornwood, N.Y.).

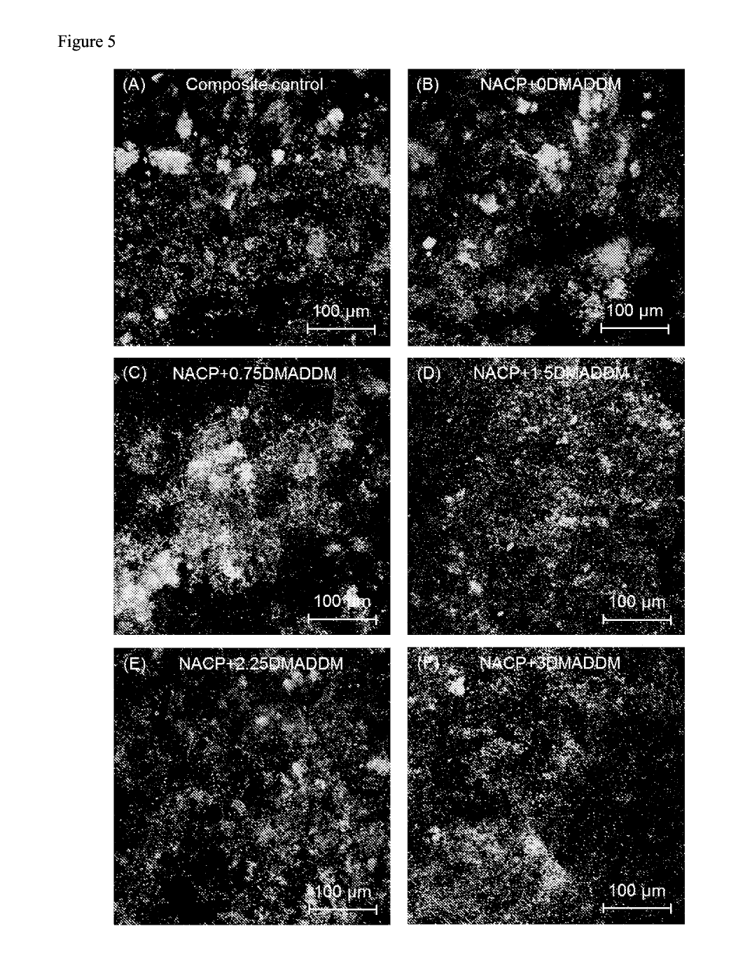

Representative CLSM images of live/dead stained biofilms adherent on the composites are shown in FIG. 5. Biofilms on composite control and NACP nanocomposite without DMADDM had primarily live bacteria. Increasing the DMADDM mass fraction in the nanocomposite resulted in much more red/yellow/orange staining, indicating that the DMADDM-containing nanocomposites effectively inhibited the biofilm growth. These results also indicate that NACP was not antibacterial, and DMADDM was responsible for the antibacterial activity.

### MTT Assays

MTT (3-(4,5-Dimethylthiazol-2-yl)-2,5-diphenyltetrazolium bromide) assay was performed according to previous studies (Antonucci J M et al. 2012; Cheng L et al. 2012a). It is a colorimetric method that measures the enzymatic reduction of MTT, a yellow tetrazole, to formazan. Briefly, disks with 48-h biofilms were rinsed with PBS and transferred to 24 well plates. Then, 1 mL of MTT dye (0.5 mg/mL MTT in PBS) was added to each well and incubated for 1 h. The disks were transferred to new 24-well plates, 1 mL of dimethyl sulfoxide (DMSO) was added to solubilize the formazan crystals, and the plate was incubated for 20 min in the dark. Then, 200 μL of the DMSO solution from each well was transferred to a 96-well plate, and the absorbance at 540 nm was measured via a microplate reader (SpectraMax M5, Molecular Devices, Sunnvale, Calif.) (Cheng L et al. 2012a).

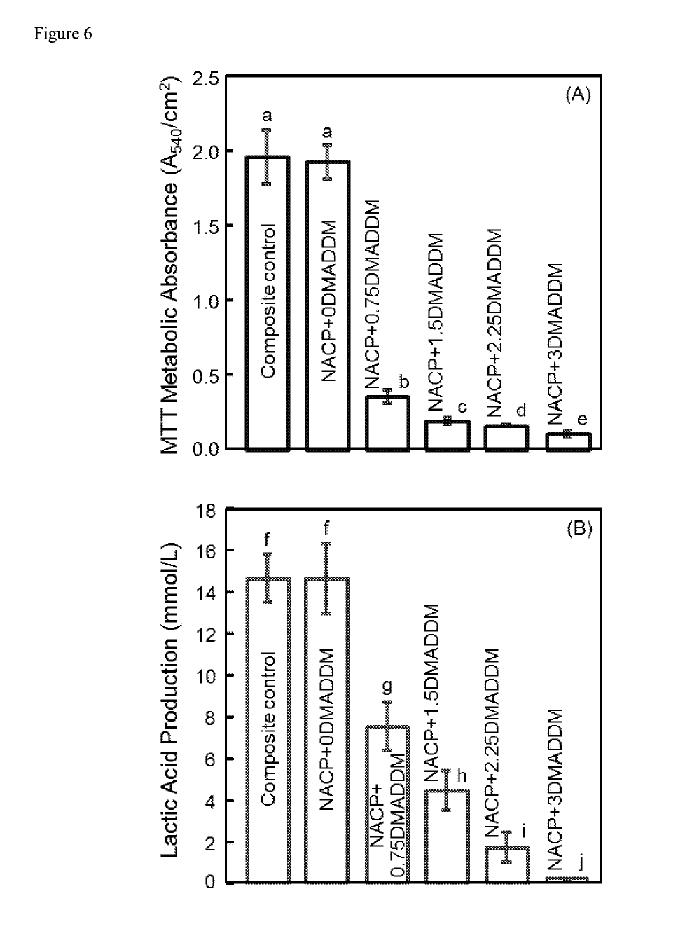

FIG. 6 plots (A) the MTT assay, and (B) lactic acid production of biofilms adherent on the composites. Each values is mean±sd (n=6). In (A), the biofilms on composite control and NACP+0% DMADDM had a similar metabolic activity (p>0.1). Increasing the DMADDM mass fraction significantly decreased the metabolic activity of biofilms (p<0.05). At 3% DMADDM in the composite, the metabolic activity was approximately 5% of that on composite control. In (B), the biofilms on composite control produced the most acid, similar to that on NACP+0% DMADDM. With increasing DMADDM mass fraction, the lactic acid production monotonically decreased (p<0.05). The lactic acid production by biofilms on NACP+3% DMADDM was about 1% of that on the commercial composite control.

### Lactic Acid Production and CFU Counts

Composite disks with 48-h biofilms were rinsed in cysteine peptone water (CPW) to remove the loose bacteria. Each disk was placed in a new 24-well plate and 1.5 mL of buffered peptone water (BPW) supplemented with 0.2% sucrose (Cheng L et al. 2012a). The samples were incubated in 5% CO₂ at 37° C. for 3 h to allow the biofilms to produce acid. The BPW solutions were then stored for lactate analysis. Lactate concentrations were determined using an enzymatic (lactate dehydrogenase) method according to previous studies (Cheng L et al. 2012a; Cheng L et al. 2012b). The microplate reader was used to measure the absorbance at 340 nm for the collected BPW solutions. Standard curves were prepared using a lactic acid standard (Supelco Analytical, Bellefonte, Pa.) (Cheng L et al. 2012a; Cheng L et al. 2012b).

Composite disks with 2-day biofilms were transferred into tubes with 2 mL CPW, and the biofilms were harvested by sonication and vortexing at the maximum speed for 20 seconds using a vortex mixer (Fisher, Pittsburgh, Pa.). Three types of agar plates were used to assess the microorganism viability after serial dilution in CPW: Mitis salivarius agar (MSA) culture plates, containing 15% sucrose, to determine total streptococci (Lima J P et al. 2009); MSA agar culture plates plus 0.2 units of bacitracin per mL, to determine mutans streptococci (Park J H et al. 2006); and Tryptic Soy Blood Agar culture plates to determine total microorganisms (Cheng L et al. 2012b). One-way analysis of variance (ANOVA) was performed to detect the significant effects of the variables. Tukey's multiple comparison test was used at a p value of 0.05.

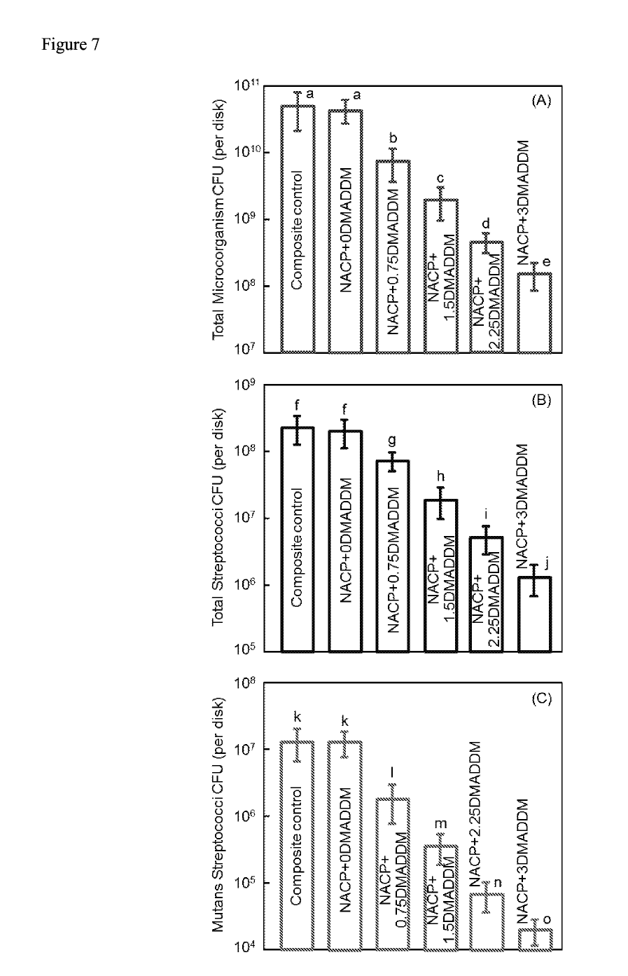

FIG. 7 plots the CFU counts for: (A) Total microorganisms, (B) total streptococci, and (C) mutans streptococci (mean±sd; n=6). The composite control had the highest CFU counts. All three CFU counts showed a similar decreasing trend with increasing DMADM mass fraction in NACP nanocomposite (p<0.05). Compared to the control composite, all three CFU counts on NACP+3% DMADDM were reduced by 2-3 orders of magnitude.

The present study demonstrated that the antibacterial monomers, such as DMADDM, could be incorporated into the NACP nanocomposite to impart a strong antibacterial activity without compromising mechanical properties. This indicates the versatility of incorporating various types of antibacterial monomers into the NACP nanocomposite, and the miscibility and compatibility of the antibacterial monomers with NACP nanocomposite. It is interesting to compare the DMADDM nanocomposite of the present study with the previous QADM nanocomposite tested by the same operator using the same procedures (Cheng et al. 2012c). The previous QADM nanocomposite reduced the MTT metoblic activity by 2-fold, compared to the same control composite (Cheng L et al. 2012c). The present study using DMADDM reduced the MTT by 20-fold. In addition, the previous QADM nanocomposite reduced the lactic acid production by 2-fold (Cheng L et al. 2012c); the present study using DMADDM reduced lactic acid by 2 orders of magnitude. Furthermore, the previous QADM nanocomposite reduced the biofilm CFU counts by 3-fold (Cheng L et al. 2012c); the present study using DMADDM reduced the biofilm CFU by 2-3 orders of magnitude. Therefore, the new DMADDM-NACP nanocomposite represents a substantial improvement over previous antibacterial dental composites.

All documents, books, manuals, papers, patents, published patent applications, guides, abstracts and other reference materials cited herein are incorporated by reference in their entirety. While the foregoing specification teaches the principles of the present invention, with examples provided for the purpose of illustration, it will be appreciated by one skilled in the art from reading this disclosure that various changes in form and detail can be made without departing from the true scope of the invention.

### CITED DOCUMENTS

Antonucci J M, Zeiger D N, Tang K, Lin-Gibson S, Fowler B O, Lin N J. Synthesis and characterization of dimethacrylates containing quaternary ammonium functionalities for dental applications. Dent Mater 2012; 28:219-228.
Bayne S C, Thompson J Y, Swift Jr. E J, Stamatiades P, Wilkerson M (1998). A characterization of first-generation flowable composites. J Am Dent Assoc 129:567-577.
Burne R A (1998). Oral Streptococci . . . Products of their environment. J Dent Res 77:445-452.
Carla A, Nikdel K, Wennerberg A, Holmberg K, Olsson J. Surface characteristics and in vitro biofilm formation on glass ionomer and composite resin (2001). Biomaterials 22:481-487.
Cenci M S, Pereira-Cenci T, Cury J A, ten Cate J M. Relationship between gap size and dentine secondary caries formation assessed in a microcosm biofilm model (2009). Caries Res 43:97-102.
Chai Z, Li F, Fang M, Wang Y, Ma S, Xiao Y, Huang L, Chen J. The bonding property and cytotoxicity of a dental adhesive incorporating a new antibacterial monomer. Oral Rehab 2011; 38:849-856.
Cheng L, Weir M D, Xu H H K, Antonucci J M, Kraigsley A M, Lin N J, Lin-Gibson S, Zhou X D. Antibacterial amorphous calcium phosphate nanocomposite with quaternary ammonium salt and silver nanoparticles. Dent Mater 2012a; 28:561-572
Cheng L, Zhang K, Melo MAS, Weir M D, Zhou X D, Xu H H K. Anti-biofilm dentin primer with quaternary ammonium and silver nanoparticles. J Dent Res 2012b; 91:598-604.
Cheng L, Weir M D, Zhang K, Xu S M, Q Chen, Zhou X D, Xu H H K. Antibacterial nanocomposite with calcium phosphate and quaternary ammonium. J Dent Res 2012c; 91:460-466.
Chow L C, Sun L, Hockey B (2004). Properties of nano structured hydroxyapatite prepared by a spray drying technique. J Res NIST 109:543-551.
Deng D M, van Loveren C, ten Cate J M (2005). Caries-preventive agents induce remineralization of dentin in a biofilm model. Caries Res 39-216-223.
Deligeorgi V, Mjör I A, Wilson N H (2001). An overview of reasons for the placement and replacement of restorations. Prim Dent Care 8:5-11.
Drummond J L, Bapna M S (2003). Static and cyclic loading of fiber-reinforced dental resin. Dent Mater 19:226-231.
Drummond J L (2008). Degradation, fatigue, and failure of resin dental composite materials. J Dent Res 87:710-719.
Featherstone J D B (2000). The science and practice of caries prevention. J Am Dent Assoc 131:887-899.
Featherstone J D B (2004). The continuum of dental caries—Evidence for a dynamic disease process. J Dent Res 83: C39-C42.
Ferracane J L (1995). Current trends in dental composites. Crit Rev Oral Biol Med 6:302-318.
Frost P M (2002). An audit on the placement and replacement of restorations in a general dental practice. Prim Dent Care 9:31-36.
Huang L, Xiao Y H, Xing X D, Li F, Ma S, Qi L L, Chen J. Antibacterial activity and cytotoxicity of two novel cross-linking antibacterial monomers on oral pathogens. Arch Oral Biol 2011; 56:367-373.
Imazato S, Torii M, Tsuchitani Y, McCabe J F, Russell R R B (1994). Incorporation of bacterial inhibitor into resin composite. J Dent Res 73; 1437-1443.
Imazato S, Kuramoto A, Takahashi Y, Ebisu S, Peters M C. In vitro antibacterial effects of the dentin primer of Clearfil Protect Bond. Dent Mater 2006; 22:527-532.
Imazato S (2003). Review: Antibacterial properties of resin composites and dentin bonding systems. Dent Mater 19:449-457.
Jokstad A, Bayne S, Blunck U, Tyas M, Wilson N (2001). Quality of dental restorations. FDI Commision Projects 2-95. International Dent J 51:117-158.
Krämer N, Garcia-Godoy F, Reinelt C, Frankenberger R (2006). Clinical performance of posterior compomer restorations over 4 years. Am J Dent 19:61-66.
Lim B S, Ferracane J L, Sakaguchi R L, Condon J R (2002). Reduction of polymerization contraction stress for dental composites by two-step light-activation. Dent Mater 18:436-444.
Lima J P, Sampaio de Melo M A, Borges F M, Teixeira A H, Steiner-Oliveira C, Nobre Dos Santos M, et al. Evaluation of the antimicrobial effect of photodynamic antimicrobial therapy in an in situ model of dentine caries. Eur J Oral Sci. 2009; 117:568-74.
Loesche W J (1986). Role of Streptococcus mutans in human dental decay. Microbiological Reviews 50:353-380.
Lu H, Stansbury J W, and C. N. Bowman C N (2005). Impact of curing protocol on conversion and shrinkage stress. J Dent Res 84:822-826.
Menschutkin, N. Z. Physik. Chemie 1890, 5, 589.
McBain A J. In vitro biofilm models: an overview. Adv Appl Microbiol 2009; 69:99-132.
Mjör I A, Moorhead J E, Dahl J E (2000). Reasons for replacement of restorations in permanent teeth in general dental practice. International Dent J 50:361-366.
Park J H, Tanabe Y, Tinanoff N, Turng B F, Lilli H, Minah G E. Evaluation of microbiological screening systems using dental plaque specimens from young children aged 6-36 months. Caries Res. 2006; 40:277-80.
Ruddell D E, M. M. Maloney M M, Thompson J Y (2002). Effect of novel filler particles on the mechanical and wear properties of dental composites. Dent Mater 18:72-80.
Sakaguchi R L (2005). Review of the current status and challenges for dental posterior restorative composites: clinical, chemistry, and physical behavior considerations. Dent Mater 21:3-6.
Sarrett D C (2005). Clinical challenges and the relevance of materials testing for posterior composite restorations. Dent Mater 21:9-20.
Stoodley P, Wefel J, Gieseke A, deBeer D, von Ohle C (2008). Biofilm plaque and hydrodynamic effects on mass transfer, fluoride delivery and caries. J Am Dent Assoc 139:1182-1190.
Svanberg M, Mjör I A, ørstavik D (1990). Mutans streptococci in plaque from margins of amalgam, composite, and glass-ionomer restorations. J Dent Res 69; 861-864.
Takahashi Y, Imazato S, Russell R R B, Noiri Y, Ebisu S (2004). Influence of resin monomers on growth of oral streptococci. J Dent Res 83:302-306.
Totiam P, Gonzalez-Cabezas C, Fontana M R, Zero D T (2007). A new in vitro model to study the relationship of gap size and secondary caries. Caries Res 41:467-473.
van Dijken J W V, Sjostrom S (1991). The effect of glass ionomer cement and composite resin filling on marginal gingival. J Clin Periodontol 18:200-203.
van Houte J (1994). Role of micro-organisms in caries etiology. J Dent Res 73:672-681.
Watts D C, Marouf A S, Al-Hindi A M (2003). Photo-polymerization shrinkage-stress kinetics in resin-composites: methods development. Dent Mater 19:1-11.
Xu H H K, Sun L, Weir M D, Antonucci J M, Takagi S, Chow L C (2006). Nano dicalcium phosphate anhydrous-whisker composites with high strength and Ca and PO₄ release. J Dent Res 85:722-727.
Xu H H K, Moreau J L, Sun L, Chow L C. Nanocomposite containing amorphous calcium phosphate nanoparticles for caries inhibition. Dent Mater 2011; 27:762-769.
Zero D T (1995). In situ caries models. Adv Dent Res 9:214-230.
Zhang K, Melo M A S, Cheng L, Weir M D, Bai Y X, Xu H H K. Effect of quaternary ammonium and silver nanoparticle-containing adhesives on dentin bond strength and dental plaque microcosm biofilms. Dent Mater 2012; 28:842-852.

1. (canceled)

2. An antibacterial resin comprising a resin and one or more antibacterial monomer,
wherein the resin is one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer; and
wherein the one or more antibacterial monomer is selected from the group consisting of dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

3. (canceled)

4. The antibacterial resin of claim 2, wherein the resin is a 1:1 mass ratio of bis-GMA and TEGDMA.

5. The antibacterial resin of claim 2, wherein the combined amount of the one or more antibacterial monomers in the antibacterial resin is a mass fraction of about 0.5% to about 50% of the antibacterial resin.

6. The antibacterial resin of claim 2, wherein the combined amount of the one or more antibacterial monomers in the antibacterial resin is a mass fraction of about 2.5% to about 20% of the antibacterial resin.

7. The antibacterial resin of claim 2, wherein the antibacterial resin further comprises one or more additional antibacterial agents selected from the group consisting of quaternary ammonium salts (QAS), silver-containing nanoparticles (NAg), chlorhexidine particles, TiO2 particles and ZnO particles.

8. The antibacterial resin of claim 2, wherein the antibacterial resin further comprises quaternary ammonium salts present at between about 1% to 30% of the mass of the antibacterial resin.

9. The antibacterial resin of claim 2, wherein the antibacterial resin further comprises silver-containing nanoparticles present at between about 0.01% and about 20% of the mass of the antibacterial resin.

10. A dental composite comprising an antibacterial resin and a filler,
wherein the antibacterial resin comprises a resin and one or more antibacterial monomer,
wherein the resin is one or more resins selected from the group consisting of bis-GM (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer; and
wherein the one or more antibacterial monomer is selected from the group consisting of dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

11. (canceled)

12. The dental composite of claim 10, wherein the resin is a 1:1 mass ratio of bis-GMA and TEGDMA.

13. The dental composite of claim 10, wherein the combined amount of the one or more antibacterial monomers in the antibacterial resin is a mass fraction of about 0.5% to about 50% of the antibacterial resin.

14. The dental composite of claim 10, wherein the combined amount of the one or more antibacterial monomers in the antibacterial resin is a mass fraction of about 2.5% to about 20% of the antibacterial resin.

15. The dental composite of claim 10, wherein the filler is one or more of a glass filler, a ceramic filler, a polymer-based filler, and nanoparticles of amorphous calcium phosphate (NACP).

16. The dental composite of claim 10, wherein the filler is a glass filler selected from the group consisting of barium boroaluminosilicate, strontium-alumino-fluoro-silicate glass, silicon dioxide, fluoroaluminosilicate glass, a ytterbium tri-fluoride filler, and a fiber glass filler.

17. The dental composite of claim 16, wherein the glass filler is barium boroaluminosilicate.

18. The dental composite of claim 10, wherein the filler is a ceramic filler selected from the group consisting of a porcelain filler, a quartz filler, or a zirconia filler.

19. The dental composite of claim 10, wherein the filler is NACP and a glass filler selected from the group consisting of barium boroaluminosilicate, strontium-alumino-fluoro-silicate glass, silicon dioxide, fluoroaluminosilicate glass, a ytterbium tri-fluoride filler, and a fiber glass filler.

20. The dental composite of claim 10, wherein the filler is NACP and barium boroaluminosilicate.

21. The dental composite of claim 10, comprising between about 1% to about 70% antibacterial resin and between about 5% to about 90% filler by mass of the composite.

22. The dental composite of claim 10, comprising between about 20% to about 40% antibacterial resin and between about 60% to about 80% filler by mass of the composite.

23. (canceled)

24. The dental composite of claim 10, comprising between about 20% to about 40% antibacterial resin, between about 40% to about 60% glass filler and between about 20% to about 40% NACP by mass of the composite.

25. (canceled)

26. The dental composite of claim 10, comprising about 30% antibacterial resin, about 50% glass filler and about 20% NACP by mass of the composite, wherein the antibacterial resin comprises; (i) about 2.5% by mass DMADDM, DMAPDM, or DMAHDM as the antibacterial monomer, (ii) about 5% by mass DMADDM, DMAPDM, or DMAHDM as the antibacterial monomer, (iii) about 7.5% by mass DMADDM, DMAPDM, or DMAHDM as the antibacterial monomer, or (iv) about 10.5% by mass DMADDM, DMAPDM, or DMAHDM as the antibacterial monomer.

27. (canceled)

28. (canceled)

29. (canceled)

30. A dental composite of claim 10, wherein the glass filler is barium boroaluminosilicate, the resin is a 1:1 mass ratio of bis-GMA and TEGDMA, and wherein the NACP particles range in size from about 10 nm to about 500 nm.

31. (canceled)
